(19) United States
(12) Patent Application Publication
(10) Pub. No.: US 2002/0106412 A1
(43) Pub. Date: Aug. 8, 2002

(54) Method and materials for controlling migration of binder liquid in a powder

(75) Inventors: Charles William Rowe, Medford, MA (US); Michael J. Cima, Winchester, MA (US); Wendy E. Pryce Lewis, Watertown, MA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Therics, Inc, Princeton, NJ (US)

(21) Appl. No.: 09/904,190

(22) Filed: Jul. 10, 2001

Related U.S. Application Data
(60) Provisional application No. 60/217,205, filed on Jul. 10, 2000.

(51) Int. Cl. A61K 9/16; A61K 9/50

(52) U.S. Cl. 424/490; 264/4

(57) ABSTRACT

A method and apparatus for controlling the migration of binder liquid in a bulk powder. The bulk powder may be deposited in a powder bed and contains at least two different substances, each in powder form. One substance gives the printed part its bulk properties, forms most of the powder, and preferably is either insoluble or not significantly soluble in the binder liquid. The other powder substance is a migration control substance. Upon interaction with the binder liquid, this substance may absorb the binder liquid and form a gel or dissolve into the binder liquid increasing viscosity thereby inhibiting binder migration. No chemical reactions occur between the binder liquid and any of the substances in the powder bed. In another embodiment of the instant invention, binder migration may be further controlled by first printing a barrier region in the powder bed containing the migration control substance. The instant invention provides functional and aesthetic advantages including more accurate release profiles in oral dosage forms and more dimensionally controlled edges and surfaces of parts. The result is sharper, more dimensionally controlled edges and surfaces of parts and sharper meetings of dissimilar binders in cases where more than one binder liquid is involved. The method is useful for printing pharmaceutical Oral Dosage Forms, attaining better control of the time release characteristics.

### BACKGROUND OF THE INVENTION

1. Technical Field

This invention relates to methods and apparatus for controlling migration of liquid in powder, and more specifically, for controlling the migration or bleeding of binder liquid during the three-dimensional printing process.

2. Description of the Related Art

Three-dimensional printing (3DP) is a process of manufacturing a three-dimensional part from powder in a layer-by-layer fashion. Layers of powder are spread or deposited and then drops of binder liquid are dispensed onto the power in a process resembling ink-jet printing. At predetermined places, powder particles are joined to each other and to other solid regions, and the process is repeated for successive layers until the desired object is created. Unbound powder supports bound regions until sufficient solidification has occurred, and later is removed. The basic process is described in U.S. Pat. No. 5,204,055.

One area of concern in 3DP has been the tendency of binder liquid to migrate in the powder before it solidifies, which is referred to as bleeding. Binder migration occurs as a result of capillary action. As a result, the geometric location of solidified power can be different from the geometric location of the places where the binder liquid was deposited. Binder migration has affected the dimensional accuracy and surface finish of printed parts.

Excessive binder migration has been undesirable for a number of manufacturing and design reasons. In printing pharmaceutical oral dosage forms (ODF), bleeding of the binder resulted in tables with rough surfaces that were aesthetically undesirable, more difficult to swallow, and friable. Furthermore, poor spatial resolution within tablets caused by binder bleeding resulted in a failure of the pharmaceutical to be released in the intended temporal pattern. The diffusion of the binder drop caused by bleeding has made various three-dimensional configurations imprecise or inconsistent. For example, in a pharmaceutical dosage form, a concentration variation yielding a pulsatile release profile may be achieved with a step function. However, bleeding of the binder resulted in a “smeared” step function such as a ramp or S-shape. The imprecise configuration has made it more difficult or impossible to achieve a step-function or pulsatile release of drug to a patient.

One attempt to control the binder-powder interaction and thus the bleeding problems was with a dissolution/resolidification process. The binder-power interactions that resulted in solidified material were described in “Microstructural Control during Three Dimensional Printing of Polymeric Medical Devices,” a PhD thesis at M.I.T. by Dr. Benjamin Wu (1998). In dissolution/resolidification the binder was a solvent that was capable of dissolving the powder to a significant concentration.

Three-dimensional printing by dissolution/resolidification is illustrated in FIG. 1. The first stage was the impact of the droplet on the powder bed, referred to as ballistic impact, in which the incoming liquid impacted the powder bed and possibly displaced some particles of powder as the droplet decelerated. This stage had been shown to occur over a period of approximately 10⁻⁴ seconds from the point of contact through complete deceleration of the droplet, for typical circumstances.

The second stage described in Dr. Wu's thesis was imbibition and drainage, in which the liquid spreads in the powder bed. The spreading of liquid occurred under the action of capillary flow or wicking, and could continue until limited by a criterion such as equalization of pressure within pores between powder particles. Dr. Wu indicated that the time duration of imbibition and drainage was milliseconds or tens of milliseconds.

The third stage described by Dr. Wu was dissolution, in which some particles dissolve in the liquid, possibly accompanied by swelling. Dr. Wu indicated that typical dissolution times are of the order of seconds.

The fourth stage described by Dr. Wu was re-precipitation, in which the solvent evaporated and a solid mass was left. Re-precipitation could involve solidification of any powder that was dissolved during stage 3, along with solidification of any solute that was originally dissolved into the binder fluid before it was dispensed. Dr. Wu indicated that this step could last seconds or tens of seconds.

In yet another type of binder-powder interaction, the dissolution step does not occur. Powder that was insoluble or not significantly soluble was placed in the binder liquid. Binding thus occurred because the binder liquid as dispensed from the printhead contained a significant concentration of a solute and when the binder liquid evaporated, the solute contained in the binder liquid remained and attached particles to each other and to other solidified material, leaving as a result particles bound together by the solidified solute to form a solid mass.

The principal influences on bleeding in the loose powder are the saturation parameter, the viscosity of the binder and the dimension of pores between powder particles. The saturation parameter indicates what fraction of the void space between particles is filled with dispensed liquid. Migration decreases as saturation parameter decreases, decreases as viscosity increases, and hydraulic conductivity, which is one of the factors influencing migration, decreases as pore size decreases. Changing the saturation parameter and changing powder size simply to change pore size for this purpose have not been available options in 3DP.

Dispensing a high viscosity binder was an available option only to a limited extent because of practical limitations on how high a viscosity liquid could be dispensed through nozzles or similar devices. Accordingly, another approach was to dispense a binder liquid having a viscosity which was suitable for dispensing, and, creating a chemical reaction when the binder liquid interacted with the powder, to bring about a state of matter having a higher viscosity. To date, this has been demonstrated only with one very specific binder, namely colloidal silica, whose viscosity is dependent on pH. Colloidal silica is fluid at alkaline conditions and is a gel at acidic conditions. Exploiting these properties of colloidal silica involved creating a chemical reaction between the binder liquid and the powder bed so that the pH of the binder liquid changed upon striking the powder bed. This chemical reaction required the binder to be colloidal silica in an alkaline condition and required acid particles to be included in the powder bed. This has been described in U.S. Pat. Nos. 5,660,621 and 5,851,465 issued to James Bredt and in his associated PhD thesis.

This technique, however, was not suitable for a drug delivery device or oral dosage form because colloidal silica was not suitable for consumption. Further, the technique imposed very specific requirements on both the binder composition and the powder bed composition.

Historically, binder liquids do not involve colloidal silica or have any significant dependence of viscosity on pH. In photographic quality ink-jet printing (two-dimensional printing), additives have sometimes been added to paper in order to absorb ink and limit spreading, but none of the complexity or additional processes of three-dimensional printing have been involved, nor has loose powder been involved.

FIG. 2 shows a tablet array printed using existing 3DP technology in which significant fluid migration or bleeding occurred. These tablets were fabricated using a powder system of 74-106 micron microcrystalline cellulose saturated to 90% saturation by a binding solution of 35 wt % sucrose in deionized water. The tablets, which according to the printing instructions were supposed to have diameters A of 11 mm and edges B spaced 2 mm apart, have all been connected by the migrating binder fluid. FIG. 2 illustrates the severe need for migration control in systems such as these.

FIG. 3 shows the theoretically calculated release of active from an eroding dosage form having varying degrees of sharpness of composition gradient. The assumed geometry of deposition of the drug is a single thin layer occupying a portion of the interior of a tablet as illustrated. The amount of drug initially present in the dosage form is held to be equal for all three cases. Three cases are presented representing no bleeding during 3DP, moderate bleeding which is defined as an assumed migration to 125% of the original drug region volume, and severe bleeding which is defined as an assumed migration to 160% of the original volume.

The release curves shown in FIG. 3 assume perfect erosion, and were calculated by stepped integration. FIG. 3 shows that as bleeding becomes more severe, the release of drug becomes more spread out in time and pulsatile release becomes impossible, a problem which to some degree has plagued all efforts to make oral dosage forms by 3DP.

### SUMMARY OF THE INVENTION

The present invention is directed toward a method of controlling migration of binder fluid with a migration control substance. The present invention includes a bulk material typically in powder form and a binder liquid. The bulk material includes a bulk powder that may be an insoluble or not significantly soluble and a migration control substance. In 3DP, the bulk material is distributed in layers in a powder bed. According to the present invention, when the binder liquid contacts the powder bed, the binder liquid is absorbed by the migration control substance or the binder liquid dissolves the migration control substance. The absorption or dissolution of the migration control substance results in a significantly increased viscosity of the binder liquid. Migration of the binder liquid is thus inhibited as a result of the non-chemical interactions initiated when the binder liquid contacts the powder bed and activates the migration control substance. This enables sharper, more dimensionally controlled edges and surfaces of parts. Controlling the migration of the binder further enables sharper meetings of dissimilar binders if more than one binder liquid is involved. Another migration control method, which is especially useful in the interiors of parts, involves printing of a barrier region which discourages binder migration in certain directions.

### BRIEF DESCRIPTION OF THE DRAWINGS

FIG. 1 illustrates four stages of binding during a dissolution/resolidification 3DP process in accordance with the prior art.

FIG. 2 illustrates actual bleeding in tablets made by 3DP by prior art methods.

FIG. 3 shows a theoretically calculated temporal history of drug release for various assumed amounts of bleeding of a dosage form manufactured by 3DP.

FIG. 4 illustrates the stages of binding in accordance with one embodiment of the present invention.

FIG. 5 illustrates another embodiment of the present invention which involves pre-printing and the use of two binder liquids.

FIG. 6 is a cross-section of a sample dosage form according to Example 1.

FIG. 7 is a scan across a digital image and the average number of fluorescent pixels over distance according to Example 1.

FIG. 8 is photographs of cornstarch grains before contact and after 10 seconds of immersion in water according to Example 1.

FIG. 9 is UV micrographs according to Example 1.

FIG. 10 is a photograph of ODF according to Example 1.

FIG. 11 is a chart of the viscosities of various E100/ethanol solutions according to Example 2.

FIG. 12 is UV micrographs according to Example 2.

FIG. 13 is a graph of digitally measured intensities of fluorescence according to Example 2.

FIG. 14 is a photograph of two binder liquid drops according to Example 3.

FIG. 15 is illustrations of multiple binder liquids according to Example 3.

FIG. 16 is illustration of ODFs according to Example 3.

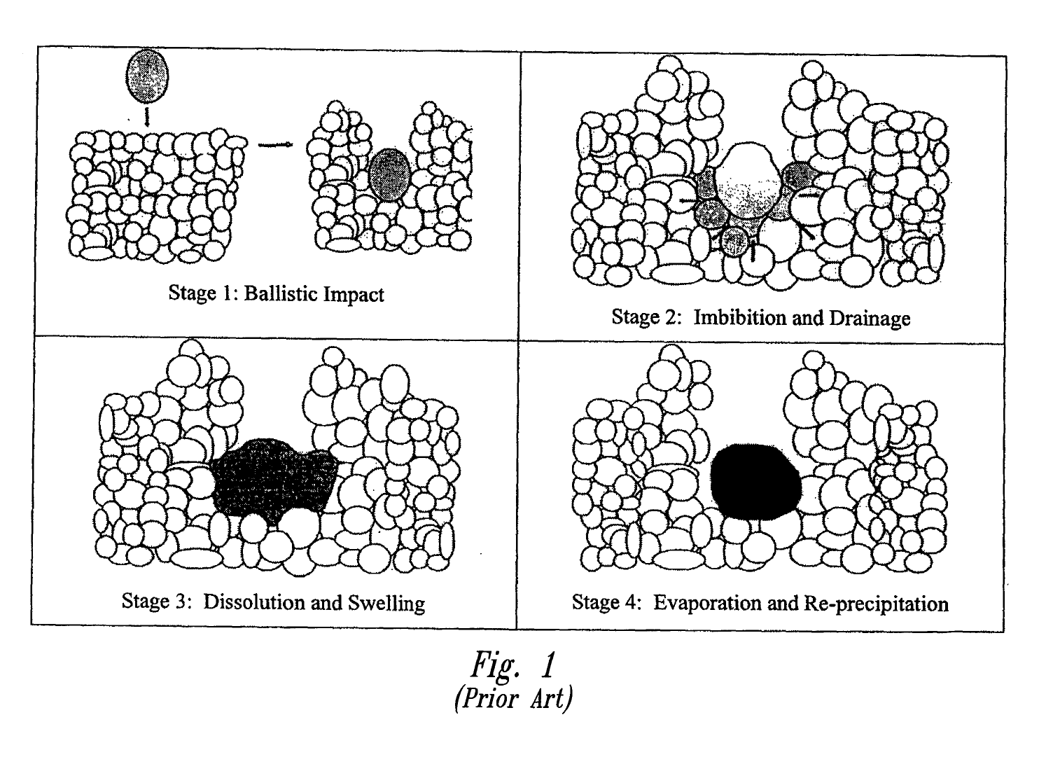

### DETAILED DESCRIPTION OF THE INVENTION

The present invention is directed toward control of binder migration in a bulk material, for example, during three-dimensional printing. The present invention includes a bulk material and a binder liquid. The bulk material includes a bulk powder and a migration control substance. The bulk powder may constitute 80% to 90% by weight of the total powder mixture. The bulk powder may be either not dissolved at all by the binder liquid or not significantly soluble in the binder liquid. The migration control substance may either absorb the binder liquid or may be dissolved by the binder liquid thus increasing the viscosity of the binder liquid.

The bulk powder substance may be either not dissolved at all by the binder liquid or not significantly soluble in the binder liquid. The phrase “not significantly soluble” is used here because there are many substances which technically have some small solubility, such as in the range of parts per thousand or less, but which are not sufficiently soluble to have any useful effect in printing. Furthermore, on a time-scale of solubility, the bulk material may not be soluble for propose of the three-dimensional printing process, but may be soluble over the longer duration of the human digestion process. If a substance is soluble in the binder liquid even up to a concentration of several weight percent such as 6 wt %, that is generally still not enough to be useful for binding by dissolution/resolidification, and so even that solubility would be considered to be not significantly soluble. Even if the bulk powder substance dissolves in the binder liquid to the extent just described, the bulk powder is considered not significantly soluble because it does not dissolve to an extent that results in any significant change to the viscosity of the binder liquid. Alternatively, the bulk powder may be soluble by the binder liquid.

In one embodiment, the migration control substance is capable of being dissolved by the binder liquid to a significant degree and thus increases the viscosity of the binder liquid upon dissolution. In another embodiment of the present invention, the migration control substance is capable of forming a gel by absorbing and swelling upon interaction with the binder liquid.

In yet another embodiment, the binder liquid may have a binding substance dissolved therein that gives the binding liquid the property of being able to bind the particles together such as by deposition of its solute around and between particles when the liquid evaporates. It is also possible for the binder liquid to be a pure solvent.

In the present invention, only a small amount, preferably less than 40%, more preferably less than 20% and most preferably 10% of the powder is the migration control substance which exhibits a gelation effect or is capable of increasing the viscosity of the liquid by dissolving into it, and the remainder of the powder in the powder bed is the bulk powder substance. Too large of a quantity of soluble particles in the powder bed may result in excessive shrinkage in the printed part.

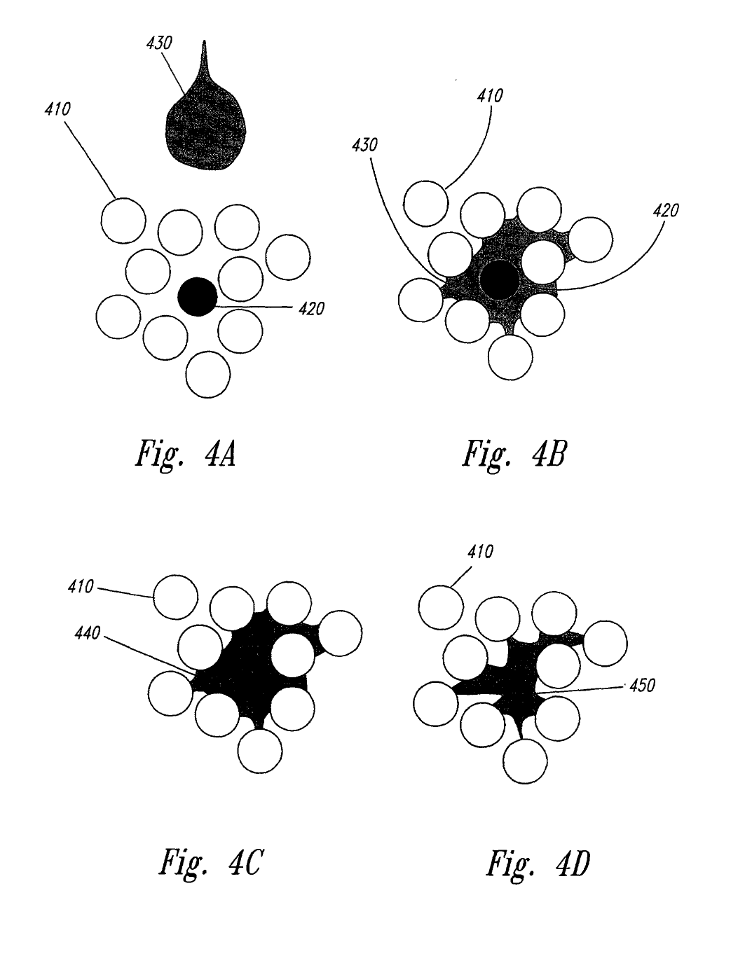

FIG. 4 illustrates one embodiment of the present invention. FIG. 4A illustrates a portion of a powder bed (not shown) comprising a plurality of particles 410 of the bulk powder material mixed in with some particles 420 of the migration control substance. FIG. 4A shows the bulk material and the binder liquid immediately prior to ballistic impact. Droplet 430 of the binder liquid approaches the powder bed containing many particles 410 of the bulk powder material and a particle 420 of a migration control substance.

FIG. 4B illustrates the imbibition and drainage stage. Droplet 430 spreads and wets a localized region of the powder bed including a plurality of bulk substance particles 410 and at least one particle 420 of the migration control substance. The time scale over which this percolation occurs depends greatly on the connectivity, the number of small pores available for infiltration at the fluid front, the available volume within the small pores, and the viscosity of the fluid. Capillary pressure motivating such transport is dependent on the packing fraction of the powder, the contact angle of the fluid with the material of the powder particles, the surface tension of the migrating fluid, and the saturation, which is the fraction of void volume which is occupied by printed fluid.

The time scale of some of the imbibition and drainage of FIG. 4B is apparently longer than estimated by the prior art. The present invention takes advantage of the fact that not all of the process of liquid spreading in the powder in the imbibition and drainage phase goes to completion in the millisecond or tens of milliseconds time period as understood and taught in the prior art. Some of the imbibition and drainage takes place within that time period, which is too short to be controlled by the process of absorption or dissolution, but it is also found that some of the spreading occurs at a sufficiently slow rate that the process of absorption or dissolution occurs and captures the spreading liquid, thus counteracting the bleeding effect.

During at least the beginning of the imbibition and drainage stage, the liquid is at high saturations, is mobile and is in what is referred to as the funicular state. At lower saturations the fluid exists as discontinuous pockets of fluid and is immobile and cannot reach from one pore to the next. This is referred to as the pendular state. Fluid reaches the immobile or pendular state when it is at a saturation of less than or approximately equal to 0.3. It is believed that at least some of the slow migration referred to here may be due to the fact that if liquid is in the funicular state and evaporation takes place at the surface where the overall wetted region adjoins the air, other liquid may migrate to that surface as replacement liquid, if the liquid properties such as viscosity allow it to migrate.

FIG. 4C illustrates the absorption or dissolution of the particle 420 of migration control substance. The droplet 430 of binder liquid combines with the migration control substance by absorbing the binder liquid forming a gel 440, or else by having the binder liquid dissolve the migration control substance particle 420 forming a high-viscosity liquid. The stage of FIG. 4C occurs simultaneous with the stage of FIG. 4B. The imbibition and drainage is inhibited by the increasing viscosity of the liquid due to the absorption or dissolution of the migration control substance.

The time for dissolution and swelling depends on the mechanism and materials properties of the substances involved. Some polymer-solvent combinations require a minimum dissolution time which has been found to be reptation limited and to depend strongly on molecular weight such as a dependence on molecular to approximately the third power.

FIG. 4D illustrates evaporation of the volatile solvent of the binder liquid. In this stage, the gel or high-viscosity liquid 440 of the stage in FIG. 4C solidifies by evaporation of solvent to form solidified mass 450. Solidified mass 450 touches many particles 410, thereby binding them together. The solidified mass 450 includes the reprecipitated solid from the migration control substance particle 420 which interacted with the binder liquid 430, along with any binding substance solute which may have been dissolved in the binder liquid 430.

The process of evaporation also competes with the process of dissolution and/or swelling because the time scales are of the same order (seconds) for the two events. The drying of a packed powder bed saturated with fluid takes place over two regimes. During an initial constant rate period, evaporation initially takes place from the external surface of the saturated region with replenishment by liquid transported to the surface from interparticle spaces by fast diffusion and capillary flow, when the liquid is in the funicular state. It is possible that this liquid motion contributes to the bleeding phenomenon. Later, the fluid becomes pendular, evaporation begins to occur at the menisci of the pores, the pores begin to become unsaturated, and the evaporation rate becomes smaller and decreases with time. The solutes within the solution begin to precipitate out of solution and deposit at the necks between particles, or in small pores within the particles themselves where the fluid meniscus remains.

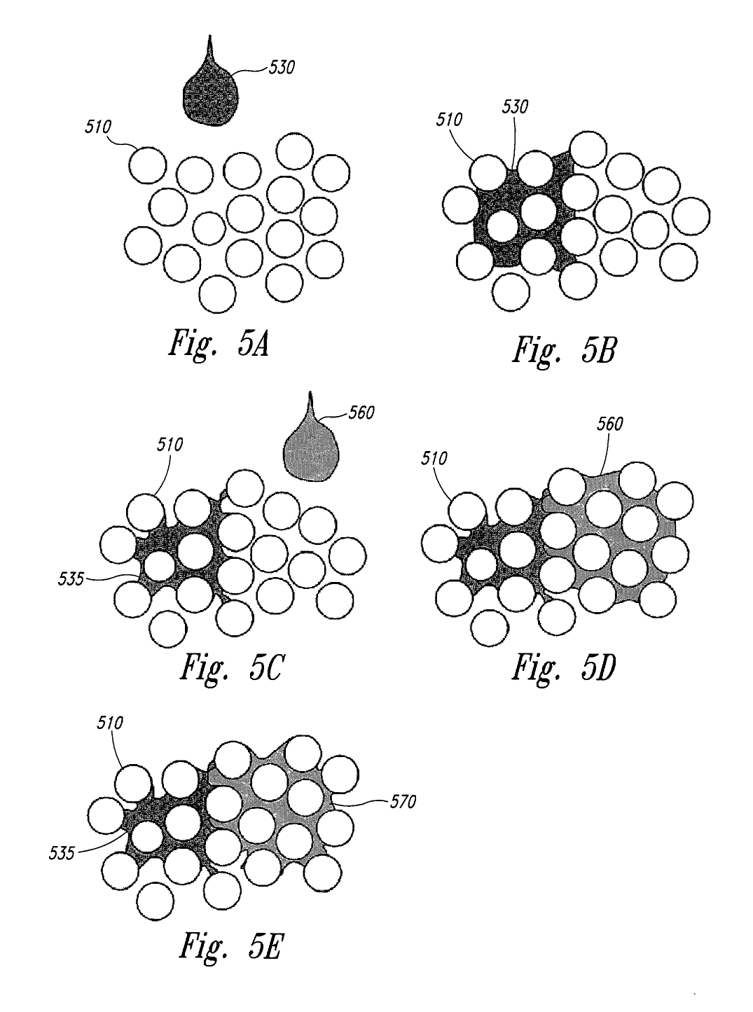

FIG. 5 illustrates yet another embodiment of the present invention. FIG. 5A shows a first drop of binder liquid 530 approaching a bed of powder particles 510. Binder liquid 530 contains a dissolved auxiliary filler substance and is of a low-migration formulation. Thus, the first binder liquid serves to specifically place a migration control substance in the powder bed. Alternatively, the binder liquid could activate a migration control substance in the bulk powder as described above.

FIG. 5B shows binder liquid 530 occupying space between powder particles in the portion of the powder bed where it has been deposited. FIG. 5C shows that after the volatile part of binder liquid 530 has evaporated, auxiliary filler substance 535 remains behind occupying somewhat less of the inter-particle void volume but still filling some of the void between particles 510, or possibly some of the volatile part of binder liquid 530 may also still be present with 535. Figure SD shows a drop of a second binder liquid 560 having been printed into a part of the powder bed adjacent to the solidified or still-liquid binder liquid 530. The migration of the drop 560 in the direction of the already-printed region is stopped by the migration barrier formed by the first binder liquid 530. Figure SE shows the second binder liquid solidified into solid mass 570.

In this embodiment of the present invention, two different binder liquids, for example, one binder liquid of low-migration and the other binder liquid of high-migration may be used to increase the sharpness of the boundary. In general, ethanolic binders exhibit less migration than aqueous binders because of the increased volatility of ethanol compared to that of water. The technique of this embodiment of the present invention is applicable to the case where the desired printing, such as of a drug-containing binder liquid, must use a relatively high-migration binder liquid such as water, but in other regions lower-migration binder liquids such as ethanol may be used. This embodiment may be further combined with the use of a migration control substance mixed in the bulk powder as described herein.

Pre-printing of certain regions with a low-migration binder such as an ethanolic binder has many advantages. One advantage is that it can be used to essentially pre-fill the pores of those regions with a solid auxiliary filler substance, preferably an auxiliary filler substance which is not very soluble in the high-migration binder liquid, so that the pores of the non-desired region do not provide much available open space for the higher-migration binder fluid to migrate into when it is printed. Yet another advantage is that if the pores of the non-desired region are already at least somewhat wet or pre-filled with low-migration binder at the time of printing the higher-migrating binder fluid, the higher-migrating binder fluid will not be able to go there.

One of the processes just identified as leading to migration control is dissolution. Dissolution is a process in which solid particles disappear into a liquid, existing therein as isolated solute molecules separated by solvent molecules. Some of the substances described herein are combinations of liquids and solutes such that the viscosity of the liquid is influenced by the amount of solute dissolved in it. For an appropriately selected combination of substances, when the solid dissolves in the liquid the resulting solution has increased viscosity compared to the binder liquid as dispensed from the printhead. The increased viscosity makes the resulting solution much less likely to spread further in the powder bed by capillary action.

In regard to the other process of migration control, some of the other states of matter formed herein are gels. A gel resembles a highly viscous liquid but is different in that for a certain range of applied stresses up to a yield stress, a gel deforms elastically and does not flow. If the stress is removed, the gel relaxes to its original state. At stresses above the yield stress, the gel behaves like a liquid. A defining feature of a gel is the presence of both the elastic region and a yield stress. Absorption is a situation where liquid molecules enter a structure of solid particles. In absorption, the solid does not totally disappear as in dissolution, but rather absorbs liquid and a gel is formed. The solid structure may expand due to the uptake of liquid, but essentially the solid molecules remain in close proximity and have some structure, which is what creates the gel.

If the migration control substance absorbs the binder liquid and forms a gel, that formation of a gel not only achieves a substantial increase in effective viscosity but also frequently results in swelling or an increase of volume. The principal effect of swelling is to decrease the dimensions of void spaces between powder particles, which is a further useful feature because smaller spaces cause lower hydraulic conductivity and therefore bleeding is reduced.

One category of binder liquids in widespread use is water-based or aqueous binders. Examples of substances which can be used as absorbers with aqueous binders are: Hydroxypropylmethyl celluloses (HPMCs), polyvinyl alcohols (PVAs), polyoxyethylene oxides, polyethylene glycols, hydrophilic silica gel (e.g., Cab-O-Sil), xantham gum, gellan gum, locust bean gum, acrylic acid polymers (e.g., Carbopols and Noveon), gelatin, sodium carboxymethyl cellulose (sodium CMC), methylcellulose (MC), guar gum, sodium alginate, polyethylene-polypropylene copolymer (Pluronics), and corn starch. Starch compounds may be used in the pregelatinized form. The above substances form gels by absorbing water. The gel-creating aqueous combination used in Example 1 is water plus cornstarch. An example with water which forms a liquid of increased viscosity is PVPs (polyvinyl pyrrolidones) with water.

Another category of binder liquids is binders which are based on ethanol or other alcohols. Examples of substances which can be used as absorbers with ethanolic binders are: Polyethylene glycols, polyethylene-polypropylene copolymers (e.g., Pluronics), polyoxyethylene alkyl ethers (Brijs, Cremophors and Plurafacs), polyvinyl pyrrolidones (PVPs). Another example of a gel created by absorbing ethanol is ethanol+HPMC. Substances which form increased-viscosity solutions with ethanol are methacrylates and methacrylic ester copolymers. The increased-viscosity ethanolic solution used in Example 2 is ethanol plus methacrylic ester copolymers.

Another category of binder liquids useful for certain applications is binders which are based on chloroform or similar halogenated hydrocarbons. Examples of substances which can be used as either absorbers or viscosity increasers with chloroform type binders are PLLA (poly-L-lactic acid) and PLGA (poly lactic co-glycolic acid) and mixtures thereof.

Examples of a binding substance which may be dissolved in binder liquid are polyacrylic acid, and, as used in Examples 1 and 2, sucrose. This binding substance, as previously described, solidifies around particles when its solute evaporates, and thereby binds those particles together.

The invention is further illustrated but is in no way limited by the following examples. These examples pertain to dispensing of drug into oral dosage forms. Experiments were conducted with both aqueous binders and alcohol based binders.

### EXAMPLE 1

### Gelation, Aqueous Binder

The basic experiment was to print a pattern onto a bed of single-component powder (bulk powder substance) which was insoluble or not significantly soluble in the binder liquid, and then for comparison to print similarly onto a bed of the same powder material which additionally contained a small fraction of a migration control powder substance. Printing was done with a continuous jet charge-and-deflect printhead as described in U.S. Pat. No. 5,807,437 and elsewhere as is known in the art. The orifice diameter was 51 microns (0.002 inch) and the drop diameter was estimated as 90 microns.

The saturation parameter, which is the volume of dispensed liquid divided by volume of empty space, is a useful descriptor of a printing process. If the saturation is greater than unity, that essentially forces some fluid out of the region of the unit cell or control volume, which is bleeding. If the saturation parameter is unity, then all of the empty space is theoretically filled with binder liquid, but in practice there would still be some bleeding. If the saturation parameter is less than unity, then there remains some empty space after the liquid has entered the interstices between the powders. There is a tradeoff, involving saturation parameter, between mechanical strength and dimensional resolution. A relatively large saturation parameter is good for mechanical strength, but a relatively large saturation parameter works against resolution (achievement of small feature size) because it encourages bleeding.

In the experimental results reported in Examples 1 and 2, the saturation parameter was 1.0, meaning the dispensed liquid exactly filled the void space between particles.

In the experimental specimens reported in Example 1 and also in Example 2 herein, all of the powder beds involved spray-dried lactose at a particle size of 74-106 microns, which means that the powder consisted of particles which fall through a sieve which passes 106 micron particles, minus the particles which fall through a sieve which passes 74 micron particles. In order to create a comparison experiment, each binder solution was printed into two types of powder beds: the just-described lactose particles, or the just-described lactose particles plus fine particles of the migration control substance.

In Example 1 the fine particles of the migration control substance mixed into the powder were cornstarch for use with the aqueous binder solution. In all cases, the particle dimensions of the migration control substance were kept quite small (less than 38 microns, which was smaller than the size of the bulk substance particles) so as to encourage especially rapid combination between the binder liquid and the migration controlling powder by either dissolution or absorption. It took approximately 2 minutes to complete one layer or print cycle before the subsequent layer was spread and printed, during which time some evaporation of the binder liquid occurred in all samples regardless of which binder liquid was used.

Following the conclusion of any printing job, the printed structures were allowed to dry for two days in a nitrogen-filled glove box. They were then set in a low-viscosity epoxy and cross-sectioned. Each of the samples was photographed under 37.5× magnification using a fluorescence microscope with a UV light source and a filter which was appropriate to view the fluorescence of the tracer substance described below. The same settings were used to image all samples.

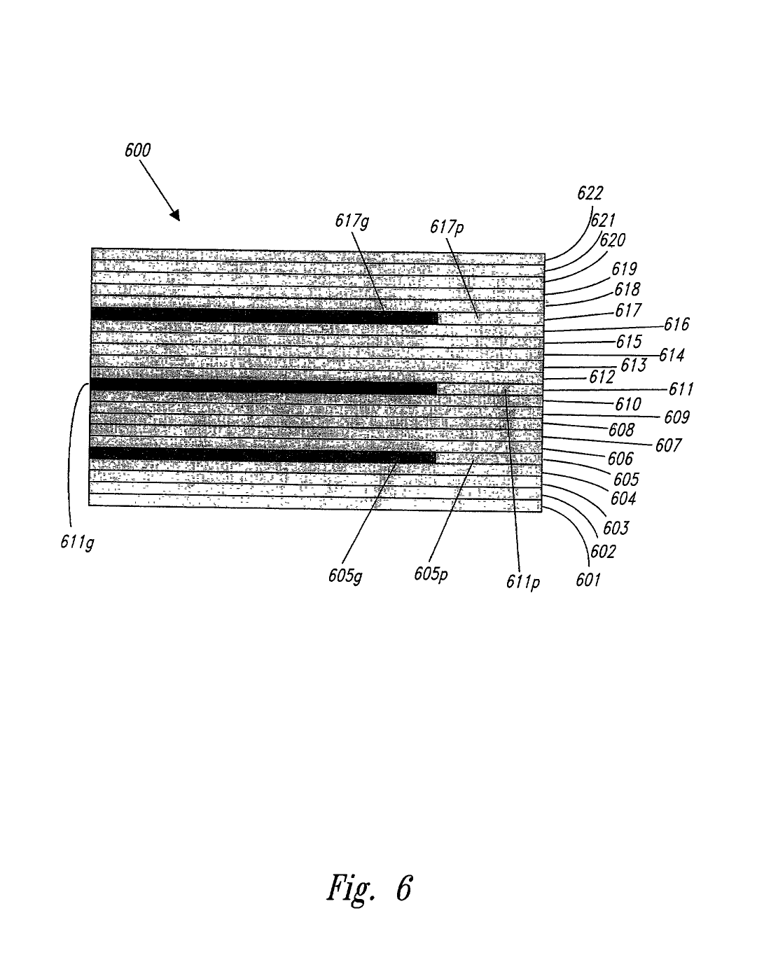

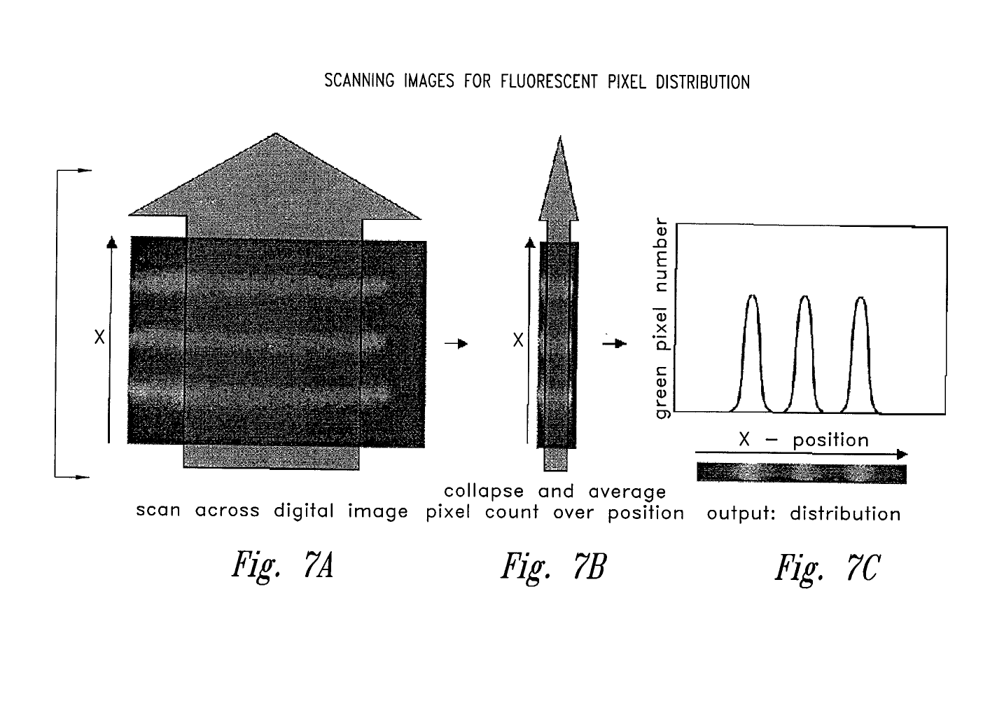

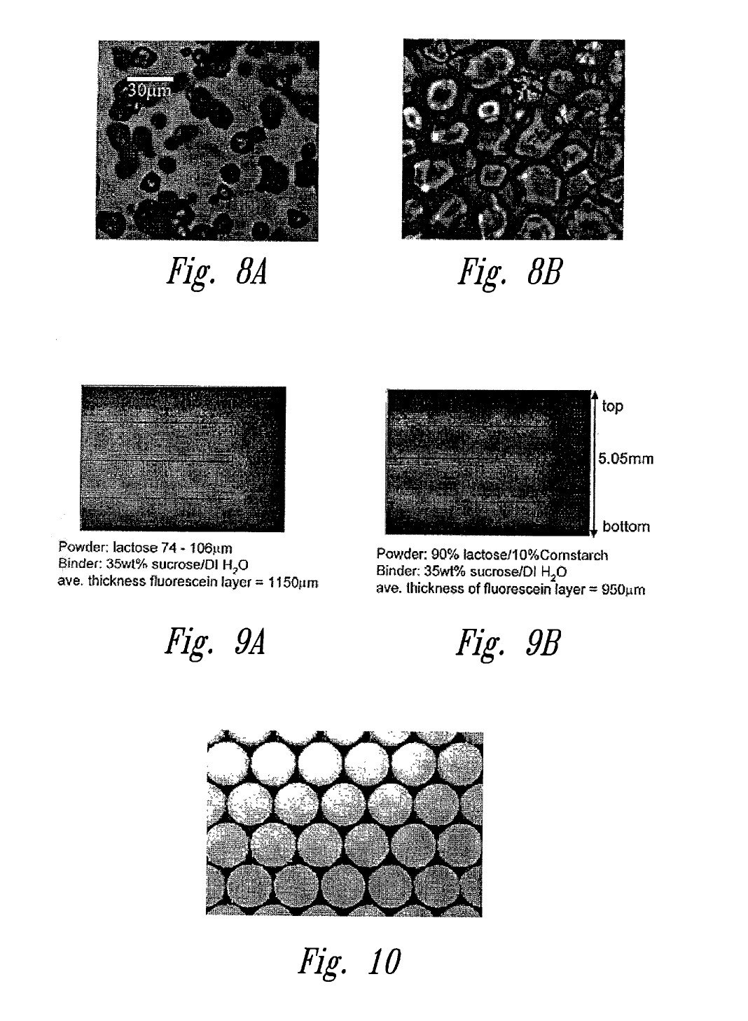

These experiments were intended to pertain to oral dosage forms containing Active Pharmaceutical Ingredients (API). As a representative experiment for Examples 1 and 2, a simple geometry containing boundaries of dissimilar regions, shown in FIG. 6, was printed, and as a surrogate for a pharmaceutical substance, these experiments used a small concentration of fluorescein, a non-pharmaceutical substance which is an easily detectable fluorescent dye. Under ultraviolet (UV) light fluorescein is visible, emitting green light. The fluorescein dissolved in the binder was deposited into parts of isolated single layers of a multi-layer printing job. At least several layers both above and below each fluorescein-containing layer were printed entirely with a similar binder containing no fluorescein tracer. FIG. 6 shows the part 600 printed for this experiment. The part was designed such that the following layers were printed only with binder fluid: 601, 602, 603, 604, 606, 607, 608, 609, 610, 612, 613, 614, 615, 616, 618, 619, 620, 621 and 622. Layers 605, 611, and 617 were printed with binder solution containing fluorescein tracer dye starting from an edge and extending most of the distance in to a certain point (regions 605g, 611g and 617g), and the rest of the way (regions 605p, 611p and 617p) they were printed with the same binder fluid as the other layers.

This stacking of layers provides an opportunity to characterize binder migration in the vertical, i.e., layer-to-layer direction. The feature that within certain layers printing of the marked binder fluid is stopped before an edge and unmarked binder fluid is used thereafter, provides an opportunity to characterize horizontal migration within a layer. The thickness of each powder layer was 225 microns. The total thickness of all 22 of the printed layers was 5.05 millimeters. The technique was calibrated by scanning UV micrographs of known fluorescein concentrations (in lactose powder) taken with the same photographic parameters, which showed that fluorescein concentration scales linearly with the intensity of fluorescent (green) pixels for the fluorescein concentrations and conditions encountered here.

The dimensions of the fluorescein features were measured by converting the optical micrograph into digital form and counting the intensity of the fluorescent (green) component of the image pixels. The fluorescent pixel intensity across the image from the bottom of the printed structure, through the fluorescein layer, to the top of the structure shows zero intensity in regions where no fluorescein was present, and reaches a peak near the center of each fluorescein layer.

Analysis of concentration of the marker substance was done by analyzing the UV micrographs for fluorescent pixel intensity using a program written to scan across the images and average the number of fluorescent (green) pixels over distance as shown in FIGS. 7A, 7B, and 7C. The output is the number of fluorescent pixels as a function of position. In photographs such as these, light-colored regions are fluorescent, indicating the deposition or spread of tracer-containing binder to them. The position and intensity of fluorescence were then compared to the intended geometric design shown in FIG. 6.

For purposes of creating a simple numerical characterization of feature width, the fluorescent feature width was defined as the Full Width at Half Maximum (FWHM) of the peaks in the intensity of fluorescent pixels displayed such as in FIG. 7. This procedure was done in the two image directions to capture fluorescein migration in the vertical direction and also in the direction parallel to the fast axis of the 3DP system. In the vertical or layer-to-layer direction, the fluorescent feature widths were then divided by the intended feature width of the printed design, which is one powder layer thickness, to give a phenomenological migration ratio:

Migration ratio=MR=fluorescent feature width÷intended design width

In the vertical direction, which is the only direction for which a dimensionless ratio is reported, the thickness of a powder layer, which was also the intended dimension of the dyed region, was 225 microns. In the lateral direction, migration distance is reported as a dimensional distance beyond the intended boundary of the printed region at which the fluorescent light intensity decreased to Half Maximum. If no bleeding occurred in the vertical direction, the fluorescein would be found only in the single layer into which it was dispensed. Any additional extent of presence of fluorescein indicates migration of binder beyond that layer and indicates a lack of sharpness of the composition gradient.

Example 1 is an aqueous example and features a migration control substance, cornstarch, which absorbs water or aqueous solutions and forms a gel. As illustration of this behavior, fine particles of cornstarch (Argo) were photographed upon initial contact with deionized water. The cornstarch grains were observed to swell to at least double in size in 10 seconds at room temperature. FIG. 8A shows these grains before contact with deionized water, and FIG. 8B shows them after 10 seconds immersed in deionized water.

In this Example, printing was done with a binder solution of 60% deionized water and 40% sucrose (by weight). The powder was spray-dried lactose at a particle size of 74-106 microns. Lactose is slightly soluble in water, but the extent of its solubility is not sufficient to achieve binding, and so another more soluble binding substance (sucrose) must be included in the binder liquid to achieve binding. Lactose does not significantly change the viscosity of water when it dissolves in water. In those experiments without a migration control substance, the powder was 100% lactose. In those experiments with a migration control substance, the powder consisted of 90% by weight of the above lactose powder and 10% by weight of powdered cornstarch of a particle size less than 38 microns.

FIG. 9A shows UV micrographs of the sandwich structures printed without gelation of an aqueous binder liquid. FIG. 9B shows UV micrographs of the sandwich structures printed with gelation of an aqueous binder. In this and similar photographs, light-colored regions are fluorescent, indicating the spread of tracer-containing binder to them. Superimposed on the photograph are solid lines identifying the region in which tracer-containing binder was intended to be placed corresponding to the pattern in FIG. 6. From visual comparison of the two photographs in FIGS. 9A and 9B, some improvement of sharpness is visible resulting from the addition of the migration control substance. Example 1 Table 1 presents more quantitative results such as the migration ratio (dimensionless) for the various sandwich structures in the vertical direction MR_{z}, and also the average migration distance in the horizontal direction beyond the side of the intended regions. The vertical migration ratios are both somewhat large, but some decrease (improvement) is obtained by the use of the cornstarch migration control substance. The horizontal migration distances show a pattern similar to the vertical migration ratios.

Example 1 Table 1

| | | Vertical | Layer | | Horizontal |
|---|---|---|---|---|---|
| | | feature width | thickness | | migration |
| Solvent | Additive | (microns) | (microns) | MR_{z} | (microns) |
| water | none | 1150 | 225 | 5.11 | 870 |
| water | cornstarch | 950 | 225 | 4.22 | 530 |

The goal of this work was to increase the sharpness of the boundary and composition gradient which would enable achievement of a sharper pulsatile release of a pharmaceutical active substance, i.e., more closely resembling a step function, as the dosage form erodes. However, it must be noted that in 3DP an extremely sharp boundary is not desirable from a structural point of view. Stitching is a term which describes the fact that some mechanical adhesion between adjacent layers results from migration of binder beyond the layer in which it is deposited. This results from bleeding and is also associated with the fact that usually a layer is not completely dry before the next layer of powder is spread over it. If there were no layer-to-layer bleeding, there would be very little mechanical strength or adhesion between adjacent layers. Therefore, in order to obtain structural cohesion a migration ratio somewhat greater than unity is desired. As a rule of thumb, it is estimated that a migration ratio of about 1.5 (minimum) is desired in order to achieve reasonable mechanical strength layer-to-layer. Thus, when data for migration ratio in the vertical direction is presented, the data of both this Example and Example 2 should not be compared against the theoretical minimum migration ratio of 1, but rather should be compared against a minimum required value for practical strength reasons which would be somewhere around 1.5.

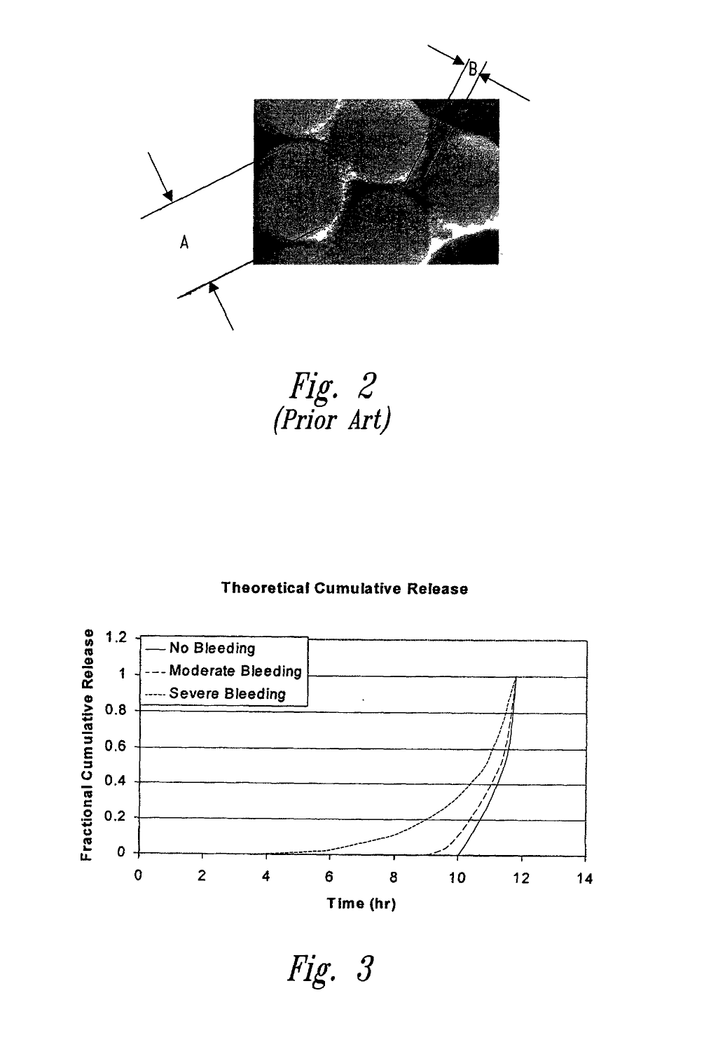

A further illustration of the improvement which is attainable by the use of these techniques is provided in FIG. 10. This figure directly corresponds to FIG. 2 which illustrated severe bleeding, except that in this case cornstarch was mixed into the powder as a migration control substance whereas for FIG. 2 no migration control substance was mixed in. The binder used was the same as used for FIG. 2, namely 35 wt % sucrose in deionized water. It can be seen that bleeding in FIG. 10 is significantly reduced compared to the bleeding in FIG. 2.

### EXAMPLE 2

### Viscosity Increase, Alcohol Based Binder

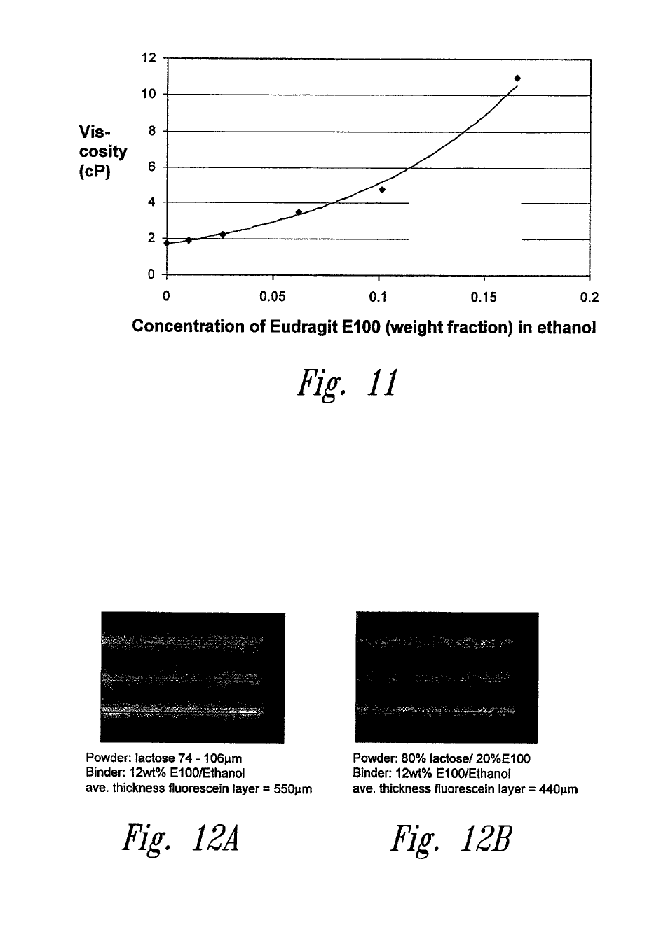

In this example, the print pattern was the same pattern used in Example 1, and the techniques were also the same. Example 2 is an ethanolic example and uses a substance, methacrylic ester copolymer Eudragit E100 (Rohm Pharma), which dissolves in and increases the viscosity of the liquid. The viscosities of E100/ethanol solutions were measured and are plotted in FIG. 11. The kinetics of dissolution of fine grains of Eudragit™ E100 and L100 at a particle size of less than 38 microns in ethanol have been observed using optical microscopy. Individual grains of both E100 (M_{w}=150,000) and L100 (M_{w}=135,000) were placed onto glass microscope slides and observed under 375× magnification. Droplets of ethanol, a good solvent for these materials, were added to the glass slides in such a way that the wetting ethanol front would interact with the grain from the side, and then surround it. Dissolution times were estimated as the time between initial contact and the point at which the particle was no longer distinguishable from the surrounding transparent medium. The dissolution of E100 and L100 grains, all less than 38 microns in diameter, took place in a duration of between 2 and 4 seconds. This is fast enough to compete with some of the wicking or spreading of liquid in the powder bed due to capillary action.

In this example, the main part of the powder again was spray-dried lactose of particle size 74-106 microns. Lactose is not significantly soluble in ethanol. The binder was ethanol containing 12 weight percent Eudragit™ E100. In experiments not using a migration control substance, the powder was 100% lactose. In experiments using a migration control substance, the additive to the powder bed was Eudragit™ E100 powder in a condition of fine particle size, <38 microns, in the proportion of 80% lactose by weight and 20% Eudragit™ E100. For this case, the photographed distribution of fluorescence intensity is given in FIGS. 12A and 12B, which may be compared to FIGS. 9A and 9B for the aqueous/gelation case. Again, lines are superimposed on the photograph to indicate the intended position of tracer-containing binder. The results are that visually, in FIGS. 12A and 12B, both regions are clearly narrower than those in FIGS. 9A and 9B, and in FIGS. 12A and 12B the spread region with the migration control substance is just slightly narrower than the spread region without the migration control substance. This example operates by the mechanism that the Eudragit™ powder mixed in with the powder bed dissolves in the ethanolic binder liquid and increases its viscosity as shown in FIG. 11. The maximum solubility of Eudragit in ethanol is 17%, at which point the viscosity is approximately double the viscosity of the dispensed solution.

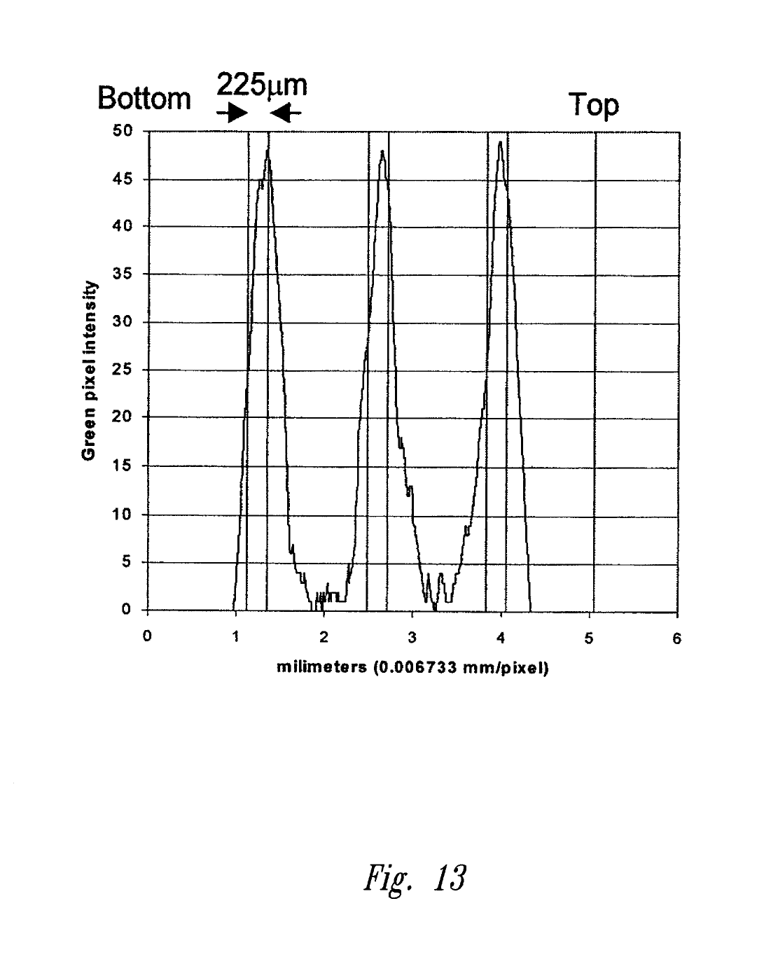

For this case of the ethanolic binder, the digitally measured intensities of fluorescence (density of fluorescent pixels) are shown in FIG. 13 for the case with the ethanol binder with E100 fines added to the powder as a migration control substance. The top of the sandwich structure is the right side of the figure, and the bottom of the sample is the left. The fluorescent pixel peaks are shown to be centered around the intended regions, but with a slight shift to the right (or top) of the structure. Such upward migration of the fluorescein in the binder may result from wicking upward into freshly spread powder.

The quantitative results, in terms of measured dimensions of the fluorescent region, are given in Example 2 Table 1. The vertical migration ratios are improved by the use of the viscosity-increasing migration control powder additive. The horizontal migration distances show a pattern of improvement similar to that of the vertical migration ratios. All of these migration ratios and migration distances are smaller (better) than those in Example 1.

Example 2 Table 1

| | | Vertical | Layer | | Horizontal |
|---|---|---|---|---|---|
| | Powder | feature width | thickness | | migration |
| Solvent | Additive | (microns) | (microns) | MR_{z} | (microns) |
| ethanol | none | 550 | 225 | 2.44 | 310 |
| ethanol | E100 | 440 | 225 | 1.95 | 190 |

In this case, the migration control substance which is added as a powder to the powder bed is the same substance which is already intended to serve as the binder substance (binding substance) and which is already dissolved in the binder liquid. In other words, the migration control substance and the binding substance are identical. This is particularly convenient and non-noticeable in the finished product because there would be no evidence in the finished product of the use of a substance solely for purpose of migration control.

The two examples which have been presented so far are Example 1 (aqueous based, gelation) and Example 2 (ethanolic based, viscosity increase). In addition, it would be possible to create the opposite combinations as well. A materials set which exhibits gelation with ethanol is hydroxypropylmethylcellulose (HPMC) with ethanol. A materials set which exhibits viscosity increase with water is polyvinyl pyrrolidone (PVP) with water.

In both Example 1 and Example 2, there is observed a reduction in migration ratio in the range of 10% to 15% for the addition of the migration control substance compared to the corresponding case where no such material is included in the powder. Regardless of the presence or absence of a migration control substance, the ethanol-based dye regions are all significantly narrower than those obtained using aqueous solutions. Migration distances with ethanol were of the order of half of those for the aqueous binder. This result is believed to be due mostly to the very different evaporation rates of the two liquids, due to their differing vapor pressures. At room temperature (25° C.), the temperature at which printing was performed, the vapor pressure of water is 3.17 kPa and the vapor pressure of ethanol is 7.9 kPa. Because the ethanol is more volatile than water it remains in the bed for a shorter period of time, or more specifically, remains in the bed in a funicular state (capable of migrating) for a shorter period of time.

Again, although the migration ratio in the vertical direction as defined here could theoretically be as small as one, in practice a value of one would not be desirable because there would be little or no stitching or mechanical strength joining adjacent layers, so an estimated minimum desirable value would be about 1.5. Achieved values of migration ratio in the vertical direction should be compared to a value of approximately 1.5, not to a value of one.

### EXAMPLE 3

### Pre-Printing Migration Barriers

This strategy derives from the results of Example 1 and Example 2 and from other data, which show that in general ethanolic binders exhibit less migration than aqueous binders probably because of the increased volatility of ethanol compared to that of water. This technique described here in Example 3 could be used on a bed of powder which contains a migration control substance as previously described in Examples 1 and 2. Example 3, however, was performed with a single substance powder bed not containing a migration control substance.

Use of this technique as just described at an external surface of a printed tablet or part would result in a readily apparent modification in the form of an added exterior shell, which for some applications may be undesirable or unacceptable. However, it is also entirely possible to use this technique at boundaries of interior regions, such as near the drug-containing layers of the previously described sandwich structure, with no readily apparent impact on the overall design of the finished product, and in so doing this technique can provide a desired and useful reduction of migration.

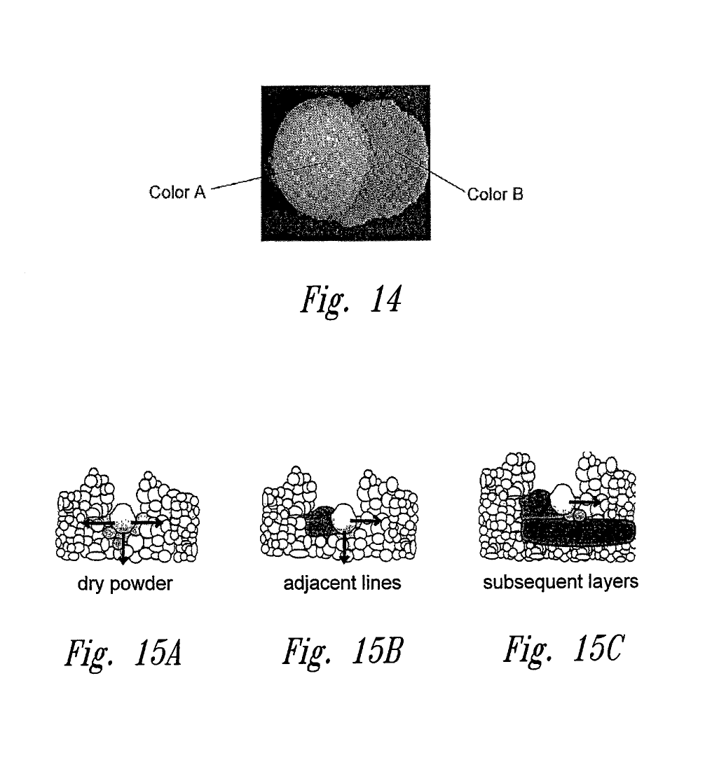

In general, powder which is saturated with a liquid exhibits lower capillary suction than unsaturated or dry powder. Given a choice of paths for migration, a fluid front would first move towards an unsaturated region, with greater capillary pull, rather than to a section that has already been saturated. A simple example of this phenomena is shown in FIG. 14 where a droplet of sugar water dyed to Color A has first been dropped into lactose powder, and then an equal sized drop of sugar water dyed to Color B is dropped onto nearly the same region. The Color A droplet saturates the powder first, stopping upon equilibration. The later-deposited Color B droplet does not migrate extensively into the already-saturated Color A region, but instead it saturates a region just outside of the Color A region.

There is also another mechanism of migration control which may be operative here. FIGS. 15A, 15B, and 15C shows how binder migration can be directed as conventionally happens during 3DP because if adjacent regions either alongside or below have been previously printed and solidified, there is less or no void space for binder liquid to migrate into those regions and so the binder liquid will migrate in other directions.

It was shown in Example 1 and Example 2 that the migration of ethanol based binder solutions is significantly smaller than that of aqueous based binder solutions. This means that it is possible to use the lesser-migrating binder, in this case the ethanolic binder, to define walls against which will later be printed the higher-migrating binder, in this case the aqueous binder. Accordingly, tablets were designed and constructed with outer wall regions, printed first with Eudragit™ L100/ethanol solution, and inner drug containing cores, printed, subsequent to the wall printing, with a naproxen aqueous suspension containing the fluorescein tracer.

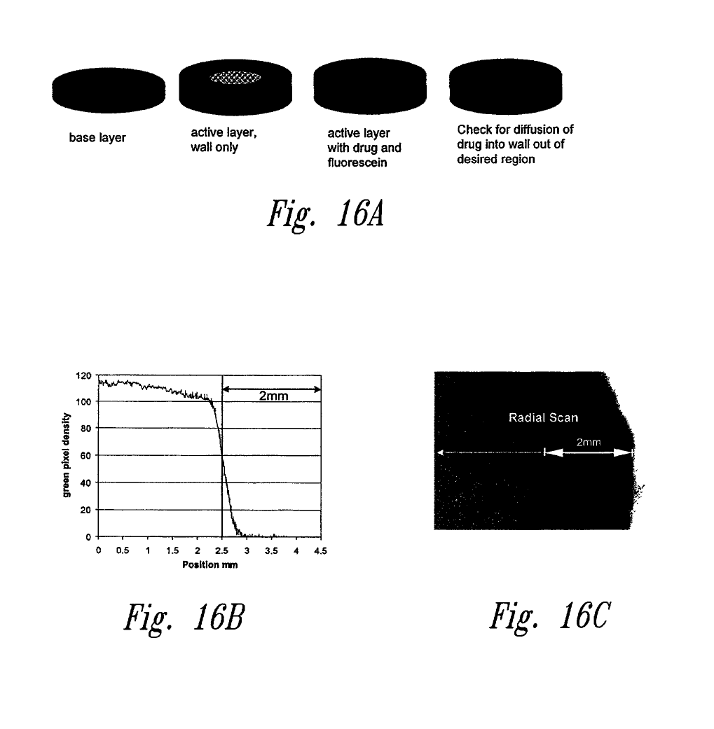

In more detail, the sequence was as follows: 1) 50% Microcrystalline Cellulose (53-74 microns)/50 wt % Lactose (53-74 microns) was spread into the layer to be printed with thickness of 200 microns. 2) Rings were printed into the powder having 11 mm outside diameter and 7 mm inside diameter using 5 wt % L100/ethanol binder solution, and allowed to dry for 2 minutes. The saturation during this first print pass was 1.0. 3) The rings were then re-printed at the same saturation to further increase the volume fraction occupied by L100 to 4.8%. 4) A circular pattern of drug-containing binder was then printed into the interior of the rings with 7 mm diameters. The drug solution in this section was a 22 wt % naproxen (Nanosystems, Inc.) suspension in deionized water+0.05 wt % fluorescein dye printed with a saturation of 1.0 for an overall Naproxen content of 10.7% of the total volume of the printed region. This procedure is illustrated in FIGS. 16A, 16B, and 16C. As in previously described sample preparation, the tablets were allowed to dry for three days in a nitrogen glove box, were set in a low-viscosity epoxy, were cross-sectioned, and were photographed under UV light. The microphotos were then scanned for intensity of fluorescent (green) pixels as previously described as a function of the radius of the circular cross-section. FIG. 16C shows the cross-section and the density of fluorescent pixels over this area.

The intensity of fluorescent pixels as a function of the radius falls off sharply at the radius which is the designed internal boundary location, indicating that there is very little migration into the printed wall region. Migration of the aqueous binder is limited to a distance of approximately 300 microns into the wall region. This can be compared to the migration in the lengthwise or horizontal direction of the aqueous binder solution out of the intended region in Example 1 for the base case (without migration control powder), which also used an aqueous binder. In that case the horizontal migration was approximately 870 microns. The powder at the time of deposition of the higher-spreading binder liquid was fairly dry due to the natural evaporation of the fairly volatile ethanol during the duration of a print cycle for one layer. However, it was not totally dry. Thus, the binder migration control mechanism illustrated by the two colored dyes of FIG. 11 was only partially operative. In addition to saturation by liquid in certain places and not others, the other mechanism believed operative here was to fill voids with the dissolved content of the low-migration binder. Because the solute remains behind upon at least partial evaporation of the solvent, the available volume for capillary imbibition decreases. Some voids in the powder were filled with the auxiliary filler substance so that there was simply less void space for the highly-migrating binder liquid to occupy. Double-printing of the pre-printing was used as just described to increase this effect.

This experiment accomplished its reduced migration at least partly because the pre-saturated wall acted to confine newly printed fluid. However, there is also yet another reason why the pre-printing operation was beneficial. The capillary pressure, which drives fluid migration, depends on the contact angle of the fluid with a particular solid substance. The contact angle of deionized water on a smooth pressed lactose surface was measured to be 30°. The contact angle of deionized water on a smooth pressed Eudragit L100 surface was measured to be 50°, which means the latter is more hydrophobic. This means that the pre-printed wall region becomes less wetting to the water than the powder particles in the designated interior region. This provides further incentive for the aqueous binder to remain in the region into which it is printed and to stay out of the pre-printed region. Although this technique is shown here used in creating a shell-like geometry involving an extra external layer, it also could be used to create internal structure which is more sharply defined than would otherwise be the case, and if used with an internal geometry there would be no separate feature required.

The advantage of the improvement which has been demonstrated in Examples 1, 2 and 3 is that in manufacturing devices containing active pharmaceutical ingredients, such as oral dosage forms, close control of drug placement can control temporal drug release patterns, can give more accurate control of dose at a predetermined time, and can result in smaller dosage forms which are more convenient to administer and have lower cost, and have enhanced reproducibility in release properties. The results of varying degrees of sharpness of concentration gradient were illustrated in FIG. 4.

Some of these applications may involve additional post-processing steps such as sintering, such as where the particles of the non-soluble substance are ceramic particles or metal particles. In addition to being used with conventional binder liquids, the present invention could be used with binder liquids which are suspensions. The present invention is usable with almost any binder liquid, in contrast to the colloidal silica gelation method which is usable only with a binder comprising colloidal silica. While the present invention does also place some requirements on composition of the powder, these requirements are not onerous and in one case do not even result in any new substances being present in the final product beyond what would have been there anyway. In particular, the present invention can produce printed parts (tablets) which are edible.

In pharmaceutical delivery devices in particular, the ability to achieve a sharp (step-function-like) composition gradient also improves the ability to achieve any other arbitrary desired composition gradient or distribution, because any other shape is mathematically equivalent to a plurality of superimposed step functions.

The method of Example 3 is especially applicable to expensive or toxic drugs as a way of minimizing waste and controlling drug placement. With its good control of drug placement, it can also be advantageous for separating, within a pill, two or more compounds which might have a reaction or an adverse effect on each other if they met.

In the description, the terms bulk powder substance, the migration control substance, and binder liquid have been used. However, any of these could be a mixture of more than one substance. A specific highly useful example is where an Active Pharmaceutical Ingredient is contained in the binder liquid. Such an ingredient could be contained as a solute in the binder liquid or, in certain cases of relatively insoluble drugs, could be contained as small solid particles suspended in the binder liquid, possibly with the aid of appropriate suspending agents and steric hindrants. Active Pharmaceutical Ingredients would most preferably be contained in the binder liquid, but could also be contained in any of the other substances.

In Example 3, the powder bed did not contain a migration control additive to the powder, such as was used in Examples 1 and 2. However, using the technique of Example 3 it would also be possible to include such an additive for further benefit.

Powder layers could be deposited by dry roller spreading, or also by other methods including slurry deposition. The printhead could be continuousjet, piezoelectric drop-on-demand, other forms of drop-on-demand, microvalve, etc., as are known in the art.

All references referred to herein are incorporated herein by reference in their entirety. Aspects of these references can be employed with the teachings of the invention to provide further combinations.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A materials set for use in 3DP by deposition of a binder liquid onto a bed of powder, wherein the powder comprises:
a bulk powder substance; and
a migration control substance distributed within the bulk powder substance, the migration control substance soluble by the binder liquid wherein the migration control substance absorbs the binder liquid and forms a gel.

2. The materials set of claim 1, wherein the migration control substance swells when it absorbs the binder liquid.

3. The materials set of claim 1, wherein the bulk powder substance, the migration control substance and the binding liquid are all edible.

4. The materials set of claim 1, wherein the binder liquid includes water and the migration control substance is selected from the group consisting of cornstarch, starch, hydroxypropylmethyl celluloses, polyvinyl alcohols, polyoxyethylene oxides, polyethylene glycols, hydrophilic silica gel, xantham gum, gellan gum, locust bean gum, acrylic acid polymers, gelatin, sodium carboxymethyl cellulose, methylcellulose, guar gum, sodium alginate, and polyethylene-polypropylene copolymer.

5. The materials set of claim 1, wherein the binder liquid includes ethanol and the migration control substance is selected from the group consisting of polyethylene glycols, polyethylene-polypropylene copolymers, polyoxyethylene alkyl ethers, polyvinyl pyrrolidones, and hydroxypropylmethylcellulose.

6. The materials set of claim 1, wherein the bulk powder substance is selected from the group consisting of lactose, other sugars, microcrystalline cellulose, hydroxypropylmethylcellulose, methacrylic ester copolymers, or a pharmaceutical excipient.

7. The materials set of claim 1, wherein the particles of the bulk powder substance comprise at least approximately 60% by weight of the powder.

8. The materials set of claim 1, wherein the particles of the migration control substance have an average particle size of less than approximately 38 microns.

9. The materials set of claim 1, wherein either the binder liquid or the bulk powder substance or the migration control substance comprises an active pharmaceutical ingredient.

10. The materials set of claim 1, wherein the binder liquid further comprises suspended solid particles.

11. A materials set for use in 3DP by deposition of a binder liquid onto a bed of powder, comprising:
liquid binder including a binding substance dissolved therein;
a bulk powder substance; and
a migration control substance intermixed with the bulk powder substance, the migration control substance dissolves in the binder liquid making a resulting solution which is more viscous than the binder liquid.

12. The materials set of claim 11, wherein the binder liquid includes water and the migration control substance is polyvinyl pyrrolidone.

13. The materials set of claim 11, wherein the binder liquid includes ethanol and the migration control substance is methacrylate or methacrylic ester copolymer.

14. The materials set of claim 11, wherein the migration control substance and the binding substance are the same substance.

15. The materials set of claim 11, wherein either the binder liquid or the bulk powder substance or the migration control substance includes an active pharmaceutical ingredient.

16. The materials set of claim 11, wherein the binder liquid further includes suspended solid particles.

17. A method of manufacturing a dosage form by 3DP, comprising:
depositing a layer of powder, wherein the powder includes particles of a bulk powder substance and particles of a migration control substance;
depositing onto the powder in selected places a binder liquid, wherein the binder liquid comprises a binding substance dissolved in it and wherein the migration control substance absorbs the binder liquid, thereby inhibiting migration of the binder liquid; and
repeating the above steps as many times as needed to manufacture the dosage forms.

18. A method of manufacturing a part by 3DP, comprising the steps of:
depositing a layer of powder wherein the powder includes particles of a bulk powder substance and particles of a migration control substance;
depositing onto the powder in selected places a binder liquid, wherein the migration control substance dissolves in the binder liquid making a resulting solution which is more viscous than the binder liquid; and
repeating the above steps as many times as needed to manufacture the part.

19. A method of controlling binder migration in three-dimensional printing, comprising:
depositing a layer of powder;
depositing a first binder liquid on the layer of powder; and
depositing a second binder liquid on the layer of powder immediately adjacent to the first binder liquid, wherein the first binder liquid provides a migration control barrier on the side adjacent to the second binder liquid.

20. The method of claim 19, wherein the first binder includes an auxiliary filler substance dissolved in it.

21. The method of claim 20, further including, allowing the first binder to substantially dry prior to depositing the second binder liquid.

22. The method of claim 19, wherein the first binder liquid includes as a solute an auxiliary filler substance which in solid form is more hydrophobic than the powder.

23. The method of claim 19, wherein the first binder liquid is an ethanol-based liquid and the second binder liquid is an aqueous liquid.

24. The method of claim 19, wherein the powder includes a migration control powder.
